(19) United States
(12) Patent Application Publication
(10) Pub. No.: US 2024/0385132 A1
(43) Pub. Date: Nov. 21, 2024

(54) Method and Apparatus for Monitoring A Vacuum Drying Process Using A Configuration of Dual MEMS Thermal Conductivity Sensor via Microcontroller

(71) Applicant: Posifa Technologies Inc., San Jose, CA (US)

(72) Inventor: Peng Tu, Palo Alto, CA (US)

(73) Assignee: Posifa Technologies Inc., San Jose, CA (US)

(21) Appl. No.: 18/197,110

(22) Filed: May 15, 2023

(51) Int. Cl. G01N 25/18; A23L 3/00; A23L 3/40; F26B 5/04; F26B 25/22

(52) U.S. Cl. CPC G01N 25/18; A23L 3/001; A23L 3/40; F26B 5/04; F26B 25/22

(57) ABSTRACT

A dual MEMS thermal conductivity sensor configuration based microcontroller apparatus for monitoring of a vacuum drying process is described. Each MEMS thermal conductivity sensor comprises a resistor and a thermopile which are laid on a hotplate suspending over a buried cavity in a silicon substrate and operates by measuring the heat lost from the hotplate to the substrate using the thermopile which is heated by applying a voltage to the resistor. One sensor is open to the environment and used for measuring the thermal conductivity of the wet air in a vacuum drying container and the other is close to the environment and filled with dry air with one atmosphere and used for offset the static output of the measurement sensor. The process monitoring includes monitoring of the partial pressure of water vapor in the vacuum drying container for controlling the water content of a dried product, monitoring the partial pressure of dry air and the total pressure in the vacuum drying container for controlling the end point of the vacuum drying process and monitoring the temperature of the vacuum drying container for protects heat sensitive product from damaging during the vacuum drying process.

### FIELD OF THE INVENTION

The present invention relates to an apparatus for simultaneous monitoring three parameters of a vacuum drying process and more particularly relates to a method for simultaneous monitoring the partial pressure of water vapor, the partial pressure of dry air and the total pressure in a vacuum drying container during a vacuum drying process using a dual Microelectromechanical systems (“MEMS”) thermal conductivity sensor configuration based microcontroller apparatus.

### BACKGROUND

Vacuum drying is a drying method that places the object to be dried in an enclosed container to vent air and reduce the pressure with a vacuum pump in order to artificially increase the water vapor partial pressure difference is called vacuum drying.

Vacuum drying is capable of reducing the flashpoint of a watery suspension from 212° F. (or 100° C., the normal boiling point of water) to just 95° F. (35° C.) and requires less heat to remove water from the product, thus using less energy and resulting in less product degradation than with traditional drying methods.

Vacuum drying has some distinctive characteristics such as higher drying rate, lower drying temperature, and oxygen-deficient processing environment. These features are beneficial and help to improve the quality and nutritional value of the dried fruits and vegetables and hence Vacuum drying has been used to dry a number of fruits/vegetables.

Vacuum drying is conceptually the ideal method for drying thermal and/or oxygen sensitive materials such as fruits and vegetables due to the advantage of removing moisture at low temperatures and minimizing the possibility of oxidation reactions. The drying kinetics and drying efficiency of vacuum drying for fruits and vegetables can be improved by monitoring or controlling one or several physical parameters.

The prior art already described means to monitor or control a vacuum drying process by monitoring one or several physical parameters as described hereinafter. One of these parameters is the product temperature, which was used in U.S. Pat. No. 5,689,895. The product temperature changes during the primary drying process and converges towards the shelf temperature. At the end of the sublimation phase (primary drying), little water (or solvent) is left and consequently the amount of chill by evaporation is reduced. By monitoring the product temperature with sensors, the end of the sublimation phase can be roughly estimated and correlated to the residual moisture in the products. However, the temperature probes influence the freeze-drying process. This can result in an early change to the secondary drying (desorption phase) which can destroy the structure of the dried product. As this test is destructive, only a few samples out of a large population (product) can be tested and one cannot ascertain that the whole population of samples (product) is sufficiently dry.

Another parameter is the pressure. During the drying process, the vacuum chamber pressure is controlled using a capacitance manometer, which measures the absolute pressure in the drying chamber. However, the Pirani vacuum gauge works on the principle of measuring the thermal conductivity of the gas in the drying chamber. The Pirani gauge reads about 60% higher than the capacitance manometer during primary drying when essentially all of the gas in the chamber is water vapor. This is because the thermal conductivity of water vapor is ˜1.6 times the thermal conductivity of nitrogen. With this inherent property, the Pirani vacuum gauge can be used to detect the end of primary drying. The point where the Pirani pressure starts to sharply decrease indicates that the gas composition is changing from mostly water vapor to nitrogen; i.e., sublimation is “essentially” complete.

Another way of using the measurement of pressure is the pressure rise test. During the pressure rise test, the vacuum chamber is completely sealed against mass transfer. The pressure difference is recorded over a defined period of time (usually several minutes). The time dependent pressure difference is the end of the secondary drying, to confirm, that the drying status of the material inside the chamber is within the specified level. Nevertheless, if a large number of items are dried, the contribution of a single item to the total pressure rise result is very small. For that reason, the test cannot identify single items or small groups of items that are not dried properly.

Still another parameter is the water vapor partial pressure inside the process gas of a freeze-drying chamber. In this case an aluminum oxide dew point sensor can be used. The Al₂O₃ sensor provides accurate determination of dew point or relative humidity in most industrial gases. The operating principle of the aluminum oxide sensor is that its capacitance varies with the moisture concentration. The Al₂O₃ sensors however suffer a major drawback since they cannot tolerate sterilizing conditions, which are a requirement for drying pharmaceuticals.

Yet another parameter is the measure of the weight of the product. In this case, balances are applied in some areas to detect weight loss of the material to be dried. In the case of pharmaceutical applications, the vials are weighed over time to determine weight loss due to the evaporating water. This method is not applicable during commercial production of clinical material, as the balances are not sterilizable. Furthermore, it is known that items directly adjacent to the balance do not dry representatively. This fact can lead to misjudgments concerning the drying state of the other items in one batch. A further disadvantage is that only a few samples out of a large population (product) can be tested.

The measurement of the water vapor has been described by U.S. Pat. No. 6,848,196 B2 as a measurable parameter for monitoring the freeze-drying process. This method involves the use of a near infrared spectrometer (NIR: Near Infrared) coupled to a light fiber to measure the residual water content of a lyophilized pharmaceutical product in situ during the process. However, the NIR-irradiation can only penetrate a few millimeters into the dried material. Therefore, a representative measurement of the entire vial is not possible. It is known that any material being adjacent to a vial can influence the drying behavior of the content of the container. Thus, the vial will not dry representatively. A further disadvantage is that only a few samples out of a large population (product) can be tested and hence a global monitoring, of the entire population cannot be achieved.

The prior arts show that the currently available for the monitoring of a vacuum drying process are not completely satisfying and still present many disadvantages.

### SUMMARY OF THE INVENTION

The objective of the invention is to overcome the inconvenience associated with the prior art and to provide an apparatus and a method which allow the monitoring of a vacuum drying process in accordance with the requirements.

As described hereinabove, the invention relates to an apparatus and a method for the monitoring a vacuum drying process comprises a dual MEMS thermal conductivity sensor configuration microcontroller apparatus. The thermal conductivity sensors widely present in the gas chromatography (GC) field; and similar sensing structures using thermopiles have been used in a variety of sensors, namely, infrared, gas flow, etc. And even closely related devices aimed to gas component quantification in binary gas mixtures.

MEMS devices offer lower power consumption and higher sensitivity than traditional mechanical counterparts simply cannot physically achieve. For instance, a MEMS thermal conductivity sensor consumes microwatts of power while offering concentration-sensitive in the 100 ppm range. This compares with the hundreds of milliwatts of power consumption for conventional thermal conductivity sensors that can only measure down to 1000 ppm at best.

The process of the invention is to use a dual MEMS thermal conductivity sensor configuration based microcontroller apparatus for simultaneous monitoring three parameters of a vacuum drying process which includes the partial pressure of water vapor, the partial pressure of dry air and the total pressure in a vacuum drying container during a vacuum drying process.

The process of the invention is much more accurate and easier to implement than the processes of the prior art because it uses a novel dual MEMS thermal conductivity sensor configuration based microcontroller apparatus which allows to cancel the offset or static output of the measurement MEMS thermal conductivity sensor.

Furthermore, because of its unique characteristics, the process of the invention allows a better monitoring and control of the vacuum drying process which leads to a safer drying process with less losses in the product which occurred with the processes of the prior art, for example because the vacuum drying was stopped too early or too late and the residual water content was too high or too lower.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a diagram showing the theoretical calculated relationship of the thermal conductivity of a binary gas mixture consisting of dry air and water vapor with the molar fraction of the water vapor in the dry air in the range of 0% to 2.4% at 24° C.;

FIG. 2 is a diagram showing the theoretical calculated relationship of the thermal conductivity of a binary gas mixture consisting of dry air and water vapor with the molar fraction of the dry air in the range of 100% to 97.6% at 24° C.;

FIG. 3 is a diagram showing the experimentally measured relationship of the thermal conductivity of a wet air with the molar fraction of the water vapor in the range of 0% to 2.4% at 24° C.;

FIG. 4 is a schematic diagram showing a vacuum drying system wherein a dual MEMS thermal conductivity sensor configuration based microcontroller apparatus is used for simultaneous monitoring the partial pressure of water vapor, the partial pressure of dry air and the total pressure in a vacuum drying container during a vacuum drying process; and

FIG. 5 is a schematic diagram showing a dual MEMS thermal conductivity sensor configuration based microcontroller apparatus for simultaneous monitoring the partial pressure of water vapor, the partial pressure of dry air and the total pressure in a vacuum drying container during a vacuum drying process.

### DETAILED DESCRIPTION

Dry air is a mechanical mixture of nitrogen, oxygen, argon and several other gases in minor amounts. It is usually modeled as a uniform (no variation or fluctuation) gas with properties averaged from the individual components.

Wet air is air that contains the highest level of water vapor. In general, air contains some moisture or water vapor, regardless of the temperature and air pressure.

Wet air can be modeled as two ideal gasses: dry air+water vapor. Since wet air is a perfect gas, the relation between the pressure P, volume V, and temperature T of a gas in the limit of low pressures and high temperatures, such that the molecules of the gas move almost independently of each other. In such a case, all gases obey an equation of state known as the ideal gas law: PV=nRT, where n is the molar fraction of the gas and R is the universal (or perfect) gas constant.

Thermal conduction in solids and ideal gases, the following formula for estimating the thermal conductivity λ of ideal gases was derived:

$$λ = 1 / 3 \cdot {c}_{v} \cdot ρ \cdot v \cdot l \qquad ( 1 )$$

In this formula c_{ν}denotes the specific heat capacity at constant volume, ρ the density of the gas, ν the mean speed of the gas molecules and ι the mean free path. Using this formula, one could assume that the thermal conductivity depends on the pressure.

For low pressures (vacuum) dependence of thermal conductivity from pressure this hypothesis is also applies. Even the pressure (particle density) in a container is reduced more and more, the particles no longer collide with each other, but rather with the container walls. At very low pressures, the mean free path is thus determined by the dimension of the container and no longer by the free path between two particle collisions. In so-called Pirani sensors, this relationship is used to draw conclusions about the pressures in a high vacuum environment on the basis of thermal conductivity.

Etim et al (Frontiers in Heat and Mass Transfer, Available at www.ThermalFluidsCentral.org) proposed a model equation for calculating the thermal conductivity of a binary gas mixture given as:

$${K}_{m i x} = 0.5 ( {k}_{A} + {k}_{B} ) / ( {N}_{A} {k}_{B} + {N}_{B} {K}_{A} ) + 0.5 ( {k}_{A} {N}_{A} + {K}_{B} + {N}_{B} ) \qquad ( 2 )$$

Where kₘᵢₓ is the thermal conductivity of a binary gas mixture, kₐ and k_{b} are the thermal conductivity of gases A and gas B, and N_{A} and N_{B} are the molar fractions of the gases A and B.

Since the sum of the mole fractions N_{A} and N_{B} is always equal to 1 so as to have an equation:

$${N}_{A} + {N}_{B} = 1 \qquad ( 3 )$$

It was shown that equation (1) gives results with good degree of accuracy. Unlike other thermal conductivity calculation models which are mostly direct functions of mole fractions, component thermal conductivities, viscosities, molar masses, densities and/or temperatures, the proposed model is just a direct function of mole fractions and component thermal conductivities.

Using equations (1) and (2), the thermal conductivity of a binary gas mixture consisting of dry air and water vapor has been calculated.

FIG. 1 is a diagram showing the calculated thermal conductivity of a wet air with water vapor in the range of 0.000 to 0.024 molar fractions at 24° C. It can be seen from the FIG. 1 that the thermal conductivity of the wet air is linear increase with water vapor fractions.

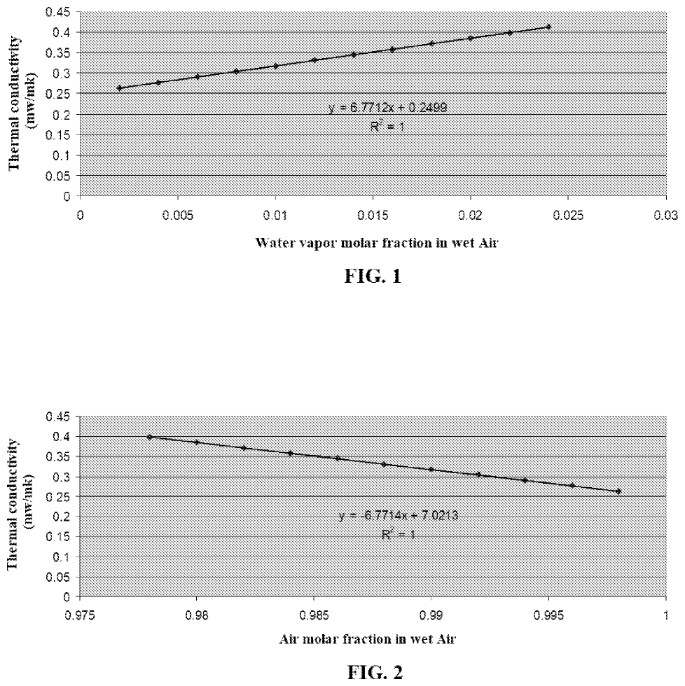

FIG. 2 is a diagram showing the calculated thermal conductivity of a wet air with dry air in the range of 0.976 to 0.998 fractions at 24° C. It also has a linear relationship between the thermal conductivity and the dry air fractions.

As shown in FIG. 3, the measured relationship of the thermal conductivity of the wet air with the water vapor molar fraction is linear proportional in the range of 0 to 0.024 molar fractions at 24° C.

All gases conduct heat to differing degrees, and the amount of heat transferred by a gas is determined by its thermal conductivity value. This property can be exploited in sensing because each gas has a different.

The thermal conductivity of a wet air can be measured using a dual MEMS thermal conductivity sensor configuration based microcontroller apparatus. A MEMS thermal conductivity sensor generally comprises a resistive heater and an integrated thermopile which are laid on a hotplate suspending over a buried cavity in a silicon substrate. The sensor operates by measuring the heat lost from a hotplate heater by a resistive heater to the silicon substrate of the sensor. The silicon substrate is thermostatically maintained at one temperature, while the heater is maintained at a higher temperature. The temperature of the hotplate depends upon heat lost to the gas with a lower temperature and filled in the buried cavity which is surrounded by a frame of the substrate. The filled gas may be a wet air consisting of water vapor and dry air. For these identical conditions, the heat loss is a function of the thermal conductivity of the wet air and the MEMS thermal conductivity sensor can convert the thermal conductivity of the wet air in the cavity into a signal output as its response.

According to the linear relationship between the thermal conductivity of wet air and its component molar fractions (water vapor and dry air) at 24° C. shown in FIG. 1, FIG. 2 and FIG. 3, a dual MEMS thermal conductivity sensor configuration based microcontroller apparatus is designed and used to monitor a vacuum drying process including the partial pressure of water vapor, the partial pressure of dry air, the total pressure and the temperature in a vacuum drying container during a vacuum drying process.

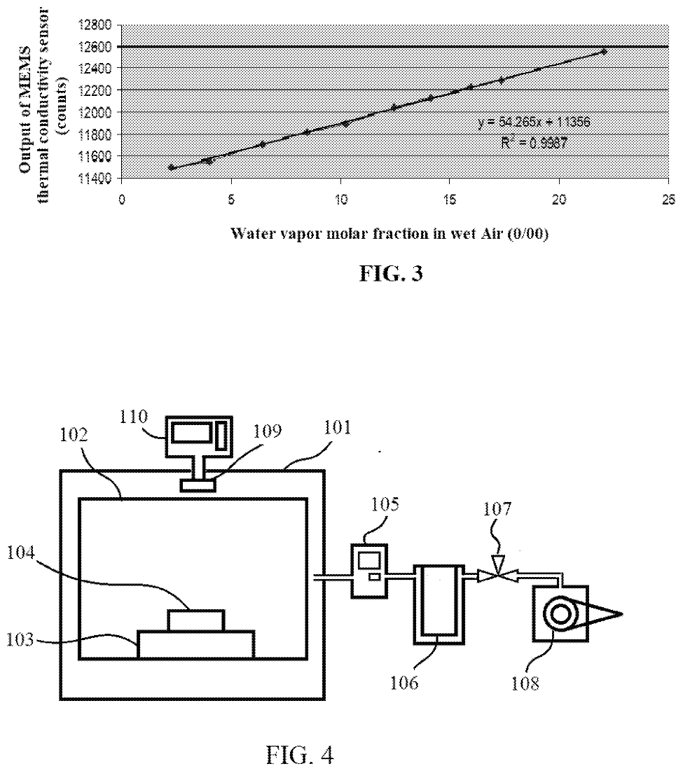

A vacuum drying system is schematically shown in FIG. 4, which consists of a vacuum drying container 101, a desiccator 102, a wire netting 103, a food sample 104, a vacuum control unit 105, a cold trap 106, a valve 107, a vacuum pump 108, a dual MEMS thermal conductivity sensor configuration based microcontroller apparatus 109 and a data logger 110. The vacuum control unit 105 is connected to the data logger so as control the vacuum of the container using a dual MEMS thermal conductivity sensor configuration based microcontroller apparatus.

The dual MEMS thermal conductivity sensor configuration based microcontroller apparatus comprises a measurement MEMS thermal conductivity sensor which is open to the environment and an offset MEMS thermal conductivity sensor which is close to the environment and filled with dry air with one atmosphere. The two MEMS thermal conductivity sensors are made using a MEMS technology and each comprises a resistor and a thermopile which are laid on a hotplate suspending over a buried cavity in a silicon substrate and operates by measuring the heat lost from the hotplate to the substrate using the thermopile which is heated by applying a voltage to the resistor.

The measurement MEMS thermal conductivity sensor is for measurement of a wet air in a vacuum drying container and the offset MEMS thermal conductivity sensor is for canceling of the offset or a static output of the measurement sensor. This is done by adjusting the heat voltage of the offset sensor and let its static output equal to the static output of the measurement sensor.

The heat voltage of the offset sensor is provided by the microcontroller which is generated based on the output of a differential amplifier amplifies the differential output of the measurement sensor and the offset sensor and its MCU senses the input data from the differential amplifier and drives its output results according to the logic written by a program.

The heat loss of the MEMS thermal conductivity sensors is mainly caused by the thermal conduction of the wet air filled in the buried cavity of the sensors. The vertical parallelepiped configuration of the cavity limits the thermal conduction from the hotplate to the bottom of the cavity in vertical downward direction which allows use one-dimensional heat transfer equation for finding the thermal conductivity of the wet air.

Since the thermal conductivity of a wet air increases proportional to the molar infractions of the water vapor and dry air in the wet air the molar infractions of the water vapor and the dry air contained in the wet air can be calculated.

According to the ideal gas law the microcontroller processes the measured data of the measurement sensor and displays the status of a vacuum drying container during a vacuum drying process.

The displayed status includes the partial pressures of the water vapor, the partial pressures of the dry air, and the total pressures in a vacuum drying container during a vacuum drying process.

The microcontroller also comprises a temperature sensor which can be used to monitor the temperature in a vacuum drying container during a vacuum drying process.

The process monitoring includes the partial pressure of the water vapor in a wet air which is used to control the water content of a dried pharmaceutical product or a dried food product during a vacuum drying process.

The process monitoring includes the total pressure in a vacuum drying container which is used to control the end point of a vacuum drying process.

The process monitoring includes the temperature which is used to protect from damaging of a heat sensitive pharmaceutical or a food product owing to a higher temperature during a vacuum drying process.

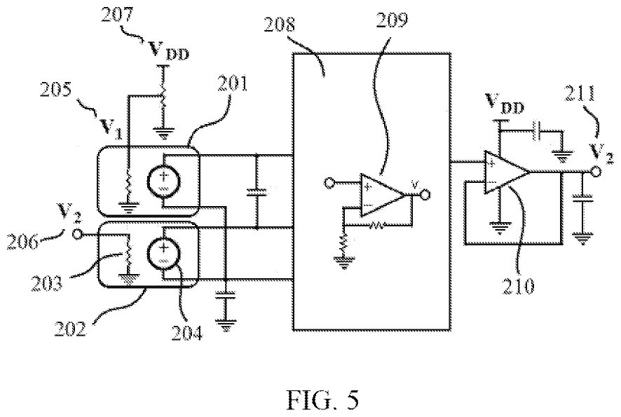

A dual MEMS thermal conductivity sensor configuration based microcontroller apparatus is shown in FIG. 5. The apparatus comprises a measurement MEMS thermal conductivity sensor 201 which is open to the environment and an offset MEMS thermal conductivity sensor 202 which is close to the environment and filled with dry air with one atmosphere. Both the sensors comprise a heating resistor 203 and a thermopile 204 for measuring the temperature of the hotplate relative to the silicon substrate. The apparatus also consists of a fixed voltage 205 for heating the resistor of the measurement sensor, an adjusted voltage 206 for heating the resistor of the offset sensor, a voltage source 207, a microcontroller 208, a differential amplifier 209 which is placed in the microcontroller, a transistor 210, and an output 211 which is generated by the microcontroller and used for adjusting the voltage 206 to be modulated as needed.

As shown in FIG. 5 there are two MEMS thermal conductivity sensors 201 and 202 wherein one is a measurement sensor for measuring the thermal conductivity of the wet air and the other is an offset sensor for canceling the offset or static output of the measurement sensor. The significant data for the thermal conductivity measurement of a wet air is the relative change of the thermal conductivity of the wet air. However, the thermopile of the measurement sensor has a relatively large value of a static output which causes a big offset in the amplifiers output and reduces the performance of the measurement system in terms of sensitivity and resolution. This offset also cannot be simply minimized by subtracting a constant value, because it depends on the ambient temperature which may vary. The static output of the offset sensor needs to be modulated by adjusting the heating voltage 206 so as to be exactly equal to the static output of the measurement sensor.

The microcontroller 208 has two main tasks: the first one is to measure the change of the thermal conductivity of the wet air and the other is to provide a heating voltage to the offset sensor 202 which is generated after the microcontroller 208 processing the output of the measurement sensor 201. The differential amplifier 209 may integrated into the microcontroller 208 and used to null its output by adjusting the heating voltage 206 of the thermal conductivity sensors 201 and 202 and then measure the change of the thermal conductivity of the wet air without any offset.

According to the linear relationship between the thermal conductivity of the wet air and the molar fractions of the water vapor and dry air we have a binary linear equation

$${V}_{output} = {b}_{air} {N}_{air} + {b}_{water} {N}_{water} + {b}_{3} \qquad ( 4 )$$

$$And$$

$${N}_{air} + {N}_{water} = 1 \qquad ( 5 )$$

In equations (4) and (5), Vₒᵤₜₚᵤₜ is an output transferred from the thermal conductivity of the wet air by the measurement of the measurement sensor and b₃ is a bias term in the sensor response, while b₁ and b₂ are the sensitivities to dry air content and water vapor content respectively. The output of the thermal conductivity sensors is expressed in the unit of Vₒᵤₜₚᵤₜ and so the coefficient bi, while the gas contents are expressed as molar fractions.

Combine equations (4) and (5) can get an equation as

$${V}_{output} = {B}_{water} {N}_{water} + {B}_{0} \qquad ( 6 )$$

$$Where$$

$${B}_{water} = {b}_{water} - {b}_{air} \qquad ( 7 )$$

$${B}_{0} = {b}_{air} + {b}_{3} \qquad ( 8 )$$

In order to find the parameters B_{water} and B₀, a set of wet air samples with known water vapor fractions needs to be measured using a MEMS thermal conductivity sensors configuration microcontroller apparatus. The measurement can be conducted in a vacuum drying system shown in FIG. 4. A capacitive humidity sensor and a capacitive pressure sensor may be used for the calibration of the measurement of the water vapor contents and the total pressure in the vacuum drying container. The wet air samples may have known water vapor contents. But under vacuum drying process the water vapor content of the wet air samples may change a little.

Having the measured data of the thermal conductivity of the wet air samples a Least Square Method can be used to find the parameters B_{water} and B₀ of equation (6) as:

$${B}_{1} = \{ ( \sum {V}_{output} ) ( \sum {{N}_{water}}^{2} ) - ( \sum {N}_{water} ) ( \sum {N}_{water} {V}_{output} ) \} / \{ ( \sum {{N}_{water}}^{2} ) - {( \sum {N}_{water} )}^{2} \} \qquad ( 9 )$$

$${B}_{0} = \{ ( \sum {N}_{water} {V}_{output} ) - ( \sum {N}_{water} ) ( \sum {V}_{output} ) \} / \{ ( \sum {{N}_{water}}^{2} ) - {( \sum {N}_{water} )}^{2} \} \qquad ( 10 )$$

The coefficient R of determination tells how strong of the linear relationship between Vₒᵤₜₚᵤₜ and N_{water} which can be found by the following equation:

$$R = \{ ( \sum {N}_{water} {V}_{output} ) - ( \sum {N}_{water} ) ( \sum {V}_{output} ) \} / \{ [ \sum {{N}_{water}}^{2} - ( \sum {V}_{output} ) ] [ ( \sum {{V}_{output}}^{2} - {( \sum {V}_{output} )}^{2} ] \} \qquad ( 11 )$$

The ideal Gas Law for a wet air can be expressed as

$${P}_{total} V = NRT \qquad ( 12 )$$

Where Pₜₒₜₐₗ is total pressure in the vacuum drying system (torr/m²),
V is volume of the vacuum drying system (m³),
N is the molar fraction of the water vapor or dry air of the vacuum drying system,
R is universal gas constant (62.37 torr/mol k)
T is absolute temperature of the vacuum drying system (k).

Substitute the molar fractions of the water vapor and the dry air of the wet air samples into equation (12) respectively, the partial pressures of the water vapor and the dry air of the measured wet air can be expressed as:

$${P}_{water} = {N}_{water} RT / V \qquad ( 13 )$$

$$And$$

$${P}_{air} = {N}_{air} RT / V \qquad ( 14 )$$

The total pressure is the sum of the dry air pressure and water vapor pressure in the vacuum drying system and can be expressed as

$${p}_{total} = {p}_{air} + {p}_{water} \qquad ( 15 )$$

Where
pₐᵢᵣ=partial pressure dry air of the vacuum drying system (torr),
p_{water}=partial pressure water vapor of the vacuum drying system (torr).

After the measured data processed the dual MEMS thermal conductivity sensor configuration microcontroller apparatus displays the partial pressures of the wet air, the partial pressure of dry air and the total pressure in the vacuum drying container during a vacuum drying process.

As described above, the MEMS thermal conductivity sensor configuration microcontroller apparatus can be used to monitor a vacuum drying process and the monitoring of the vacuum drying process includes the monitoring of the partial pressure of the water vapor in a vacuum drying container which is used to control the water content of a dried pharmaceutical product or a dried food product.

The monitoring of a vacuum drying process also includes the monitoring of the partial pressure of dry air and the total pressure in a vacuum drying container which is used to control the end point of the vacuum drying process.

The process monitoring still includes the monitoring of the temperature in a vacuum drying container which is used to protect from damaging of a heat sensitive a pharmaceutical or a food product owing to a higher temperature during a vacuum drying process.

Thus, an apparatus for monitoring a vacuum drying process has been disclosed. It is to be understood that the above-described embodiments are merely illustrative of some of the many specific embodiments that represent applications of the principles discussed above. Clearly numerous and other arrangements can be readily devised by those skilled in the art without departing from the scope of the invention.

What is claimed is:

1. An apparatus for monitoring a vacuum drying process comprises a dual MEMS thermal conductivity sensor configuration consisting of a measurement MEMS thermal conductivity sensor and a offset MEMS thermal conductivity sensor and a microcontroller consisting of a differential amplifier used to null the differential input of the measurement sensor and the offset sensor and generating a adjusted heating voltage to the offset sensor so as to provide a static output for compensating the static output of the measurement sensor and the microcontroller further adopted to monitor the vacuum drying process using the data measured by the measurement sensor which includes simultaneous monitoring of the partial pressure of water vapor, the partial pressure of dry air, the total pressure and the temperature in the vacuum drying container during the vacuum drying process.

2. The apparatus according to claim 1, wherein a measurement MEMS thermal conductivity sensor is open to the enlivenment and the other offset MEMS thermal conductivity offset is close to the enlivenment and filled with dry air with one atmosphere.

3. The apparatus according to claim 1, wherein each MEMS thermal conductivity sensor comprises a resistor and a thermopile which are laid on a hotplate suspending over a buried cavity in a silicon substrate and operates by measuring the heat lost from the hotplate to the substrate using the thermopile which is heated by applying a voltage to the resistor.

4. The apparatus according to claim 1, wherein the heat lost is mainly caused by the thermal conduction of the wet air filled in the buried cavity of the sensors.

5. The apparatus according to claim 1, wherein the heat lost measurement allows calculating the thermal conductivity of the wet air by solving a one-dimensional thermal conduction equation based on the vertical parallelepiped configuration of the buried cavity.

6. The apparatus according to claim 1, wherein the thermal conductivity of the wet air increases proportional to the molar infractions of the water vapor and dry air contained in the wet air so as to allow calculating the molar infractions of the water vapor and the dry air contained in the wet air.

7. The apparatus according to claim 1, wherein the microcontroller collects and processes the measured data of the measurement sensor and displays the process status during the vacuum drying process.

8. The apparatus according to claim 1, wherein the displayed vacuum process includes the partial pressures of water vapor, the partial pressures of dry air, and the total pressures in the vacuum drying container during the vacuum drying processes.

9. The apparatus according to claim 1, wherein the microcontroller further comprises a temperature sensor which is used to monitor the temperature in the vacuum drying container during the vacuum drying processes.

10. The apparatus according to claim 1, wherein the process monitoring includes monitoring of the partial pressure of the water vapor in the vacuum drying container which is used to control the water content of a dried pharmaceutical product or a dried food product during the vacuum drying process.

11. The apparatus according to claim 1, wherein the process monitoring includes monitoring of the partial pressure of dry air and the total pressure in the vacuum drying container which is used to control the end point of the vacuum drying process.

12. The apparatus according to claim 1, wherein the process monitoring includes monitoring of the temperature in the vacuum drying container which is used to protect from damaging of a heat sensitive pharmaceutical or food product owing to a higher temperature during the vacuum drying process.

13. A method of monitoring a vacuum drying process characterized by the steps of
Preparing a set of wet air samples each consisting of a known molar fraction water vapor and a known molar fraction dry air;
Measuring the thermal conductivity of each wet air sample in a vacuum drying system using a dual MEMS thermal conductivity sensor configuration based microcontroller apparatus;
Creating linear regression equations based on the measured data of the thermal conductivities of the wet air samples;
Selecting a best linear regression equation for describing the relationship between the thermal conductivity and the water vapor molar fraction and the dry air molar fraction;
Measuring the thermal conductivity of an unknown molar fraction of a wet air in the same vacuum drying container using the same dual thermal conductivity sensor configuration based microcontroller apparatus;
Substituting the measured thermal conductivity into the selected linear regression equation and finding the molar fractions of the water vapor and the dry air of the wet air in the vacuum drying container by solving the selected regression equation; and
Finding and at the same time displaying the partial pressure of the water vapor, the partial pressure of the dry air, the total pressure and the temperature in the vacuum drying container based on the measured data using the dual MEMS thermal conductivity sensor configuration based microcontroller apparatus.

14. The method according to claim 13, wherein the dual MEMS thermal conductivity sensor configuration comprises a measurement MEMS thermal conductivity sensor which is open to the enlivenment and an offset MEMS thermal conductivity sensor which is close to the enlivenment and filled with dry air with one atmosphere.

15. The method according to claim 13, wherein each MEMS thermal conductivity sensor comprises a resistor and a thermopile which are laid on a hotplate suspending over a buried cavity in a silicon substrate and operates by measuring the heat lost from the hotplate to the substrate using the thermopile which is heated by applying a voltage to the resistor.

16. The method according to claim 13, wherein the heat lost is mainly caused by the thermal conduction of the wet air filled in the buried cavity of the sensors.

17. The method according to claim 13, wherein the heat lost measurement allows calculating the thermal conductivity of the wet air by solving a thermal conduction equation.

18. The method according to claim 13, wherein the thermal conductivity of the wet air increases proportional to the molar infractions of the water vapor and dry air contained in the wet air so as to allow calculating the molar infractions of the water vapor and the dry air contained in the wet air.

19. The method according to claim 13, wherein the microcontroller collects and processes the measured data of the measurement sensor and displays the process status in the vacuum drying container during the vacuum drying processes.

20. The method according to claim 13, wherein the vacuum drying process includes the partial pressures of water vapor, the partial pressures of dry air, and the total pressures in the vacuum drying container during the vacuum drying processes.

21. The method according to claim 13, wherein the microcontroller further comprises a temperature sensor which is used to monitor the temperature in the vacuum drying container during the vacuum drying processes.

22. The method according to claim 13, wherein the process monitoring includes monitoring of the partial pressure of the water vapor in the vacuum drying container which is used to control the water content of a dried pharmaceutical product or a dried food product during the vacuum drying process.

23. The method according to claim 13, wherein the process monitoring includes monitoring of the partial pressure of dry sir and the total pressure in the vacuum drying container which is used to control the end point of the vacuum drying process.

24. The method according to claim 13, wherein the monitoring includes monitoring of the temperature in the vacuum drying container which is used to protect from damaging of a heat sensitive pharmaceutical or food product owing to a higher temperature during the vacuum drying process.
